(19) United States
(12) Patent Application Publication
(10) Pub. No.: US 2019/0385716 A1
(43) Pub. Date: Dec. 19, 2019

(54) MEDICAL INFORMATION DISPLAY SYSTEM

(71) Applicant: Konica Minolta, Inc., Chiyoda-ku (JP)

(72) Inventor: Kenichirou SUZUKI, Mitaka-shi (JP)

(73) Assignee: Konica Minolta, Inc., Tokyo (JP)

(21) Appl. No.: 16/438,841

(22) Filed: Jun. 12, 2019

(30) Foreign Application Priority Data
Jun. 13, 2018 (JP) 2018-112394

(51) Int. Cl. G16H 10/60; G06F 21/62; G16H 30/20

(52) U.S. Cl. CPC G16H 10/60; G16H 15/00; G16H 30/20; G06F 21/6254

(57) ABSTRACT

A medical information display system includes the following. A server includes a storage which stores first medical information and second medical information added to the first medical information. A main terminal includes a main display and a first hardware processor which obtains the first medical information from the server to display the first medical information on the main display. A supplementary display apparatus includes a supplementary display which can be worn by the user and which can be confirmed by sight by the user wearing the supplementary display apparatus and a second hardware processor which obtains the second medical information related to the first medical information displayed on the main display from the server to display the second medical information on the supplementary display.

### BACKGROUND

### Technological Field

The present invention relates to a medical information display system.

### Description of the Related Art

In addition to medical images for diagnosis obtained by imaging a subject, medical information includes personal information (for example, name, past diagnostic result, etc.) regarding the subject incidental to the medical image.

Medical information, especially personal information leaking out to a third party other than the subject and the medical worker is a serious problem. In the past, various techniques are proposed to prevent such leakage.

For example, Japanese Patent Application Laid-Open Publication No. 2010-264107 describes a technique to manage the patient information separately from the corresponding image data, and when the image data is obtained, the patient information corresponding to the image data is called from the server based on the image ID specific to the image data to be displayed on the terminal apparatus.

However, although the technique described in Japanese Patent Application Laid-Open Publication No. 2010-264107 can prevent leakage of the patient information when the image data is transferred through the network, the corresponding patient information is displayed when the medical image based on the transferred image data is displayed on the terminal apparatus.

Further, the technique described in Japanese Patent Application Laid-Open Publication No. 2010-264107 is for reading a medical image outside a medical facility, and the terminal apparatus is used outside the medical facility where there are a large number of unspecified people. Therefore, depending on where the image is read, the medical image and the patient information displayed on the terminal apparatus may be seen by a third party behind the user (physician, etc.).

Compared to inside the medical facility, the possibility that the terminal apparatus may be stolen increases outside a medical facility where there are a large number of unspecified people. When the terminal apparatus is stolen, the medical image and the patient information may be exposed to a third party.

### SUMMARY

The purpose of the present invention is to provide a medical information display system with which a user can perform diagnosis without leaking to a third party medical information which a patient does not want third parties to know even outside a medical facility where there are a large number of unspecified people.

To achieve at least one of the above-mentioned objects, according to an aspect of the present invention, a medical information display system reflecting one aspect of the present invention includes, a server which includes a storage which stores first medical information and second medical information added to the first medical information; a main terminal which includes a main display and a first hardware processor which obtains the first medical information from the server to display the first medical information on the main display; and a supplementary display apparatus which includes a supplementary display which can be worn by the user and which can be confirmed by sight by the user wearing the supplementary display apparatus and a second hardware processor which obtains the second medical information related to the first medical information displayed on the main display from the server to display the second medical information on the supplementary display.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a schematic structure diagram showing a medical information display system according to a first embodiment of the present invention.

FIG. 2 is a block diagram showing a specific configuration of a server included in the medical information display system shown in FIG. 1.

FIG. 3 is a diagram showing an example of a display image displayed on a main display of a main terminal included in the medical information display system shown in FIG. 1.

FIG. 4 is a diagram showing an example of the display image displayed on the main display of the main terminal included in the medical information display system shown in FIG. 1.

FIG. 5 is a diagram showing an example of a display position determining table stored in a server shown in FIG. 2.

FIG. 6 is a diagram showing another example of the display position determining table stored in the server shown in FIG. 2.

FIG. 7 is a block diagram showing a specific configuration of a main terminal included in the medical information display system shown in FIG. 1.

FIG. 8 is a diagram showing an example of a three-dimensional information transmitter included in the main terminal shown in FIG. 7.

FIG. 9A, FIG. 9B, and FIG. 9C show a diagonal view showing an example of a supplementary display apparatus included in the medical information display system shown in FIG. 1.

FIG. 10 is a block diagram showing a specific configuration of the supplementary display apparatus shown in FIG. 9A, FIG. 9B, and FIG. 9C.

FIG. 11A and FIG. 11B are schematic diagrams describing an example of a method that the supplementary display apparatus shown in FIG. 9A, FIG. 9B, and FIG. 9C generates spatial information.

FIG. 12 is a schematic diagram describing another example of a method that the supplementary display apparatus shown in FIG. 9A, FIG. 9B, and FIG. 9C generates spatial information.

FIG. 13A and FIG. 13B are diagrams showing how the main terminal can be viewed by the user wearing the supplementary display apparatus.

FIG. 14A and FIG. 14B are diagrams showing how the main terminal can be viewed by the user wearing the supplementary display apparatus.

FIG. 15 is a diagram showing how the main terminal is viewed by the user wearing the supplementary display apparatus.

FIG. 16 is a schematic configuration of a medical information display system according to a second embodiment of the present invention.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

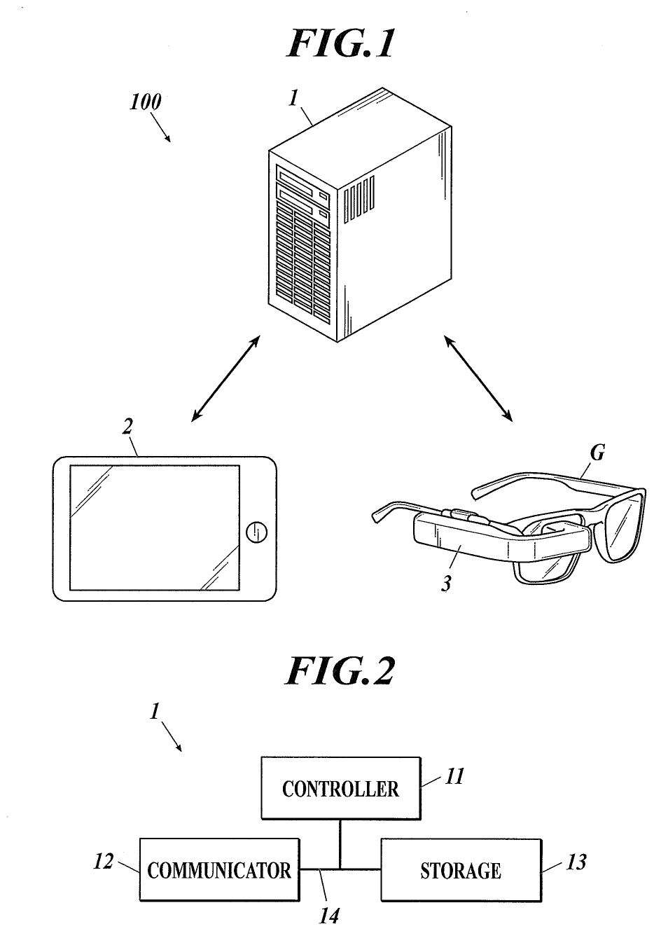

First, the first embodiment of the present invention is described with reference to FIG. 1 to FIG. 15.

[Medical Information Display System]

First, a schematic configuration of a medical information display system (hereinafter referred to as a display system 100) according to the present embodiment is described. FIG. 1 is a block diagram showing a schematic configuration of the display system 100.

As shown in FIG. 1, the display system 100 according to the present embodiment includes a server 1, a main terminal 2, and a supplementary display apparatus 3.

These are connected through a network (not shown).

The display system 100 is able to obtain data of the medical image and examination information from various modalities not shown (computer tomographic imaging apparatus (CT), magnetic resonance imaging diagnostic apparatus (MRI), digital X-ray imaging apparatus (DR), computer radiography (CR), angiography X-ray diagnostic apparatus (XA), and ultrasound diagnostic apparatus (US), etc.).

The server 1 includes a PC or a dedicated apparatus.

The server 1 can be communicated with a main terminal 2 and a supplementary display apparatus 3.

The server used in a picture archiving and communication system (PACS) can be used as the server 1 of the display system 100.

The details of the server 1 is described later.

The main terminal 2 includes a PC or a tablet terminal, etc.

The main terminal 2 can be communicated with the server 1 and the supplementary display apparatus 3.

Preferably, the main terminal 2 is portable, and is able to communicate wirelessly with the server 1 and the supplementary display apparatus 3.

The details of the main terminal 2 are described later.

The user is able to wear the supplementary display apparatus 3.

The supplementary display apparatus 3 is able to communicate with the server 1 and the main terminal 2.

Preferably, the supplementary display apparatus 3 is able to communicate wirelessly with the server 1 and the main terminal 2.

The details of the supplementary display apparatus 3 is described later.

According to such display system 100 of the present embodiment, the medical information stored in the server 1 can be viewed using the main terminal 2 and the supplementary display apparatus 3.

[Server]

Next, the specific configuration of the server 1 included in the display system 100 is described. FIG. 2 is a block diagram showing a specific configuration of the server 1.

As shown in FIG. 2, the server 1 includes a controller 11, a communicator 12, and a storage 13.

These are connected to each other through a bus 14.

The controller 11 includes a central processing unit (CPU), a random access memory (RAM), and the like. The CPU of the controller 11 reads various programs stored in the storage 13 and deploys the program in the RAM. The various processes are executed according to the deployed program. The operation of each unit of the server 1 is centrally controlled.

The communicator 12 includes a wireless module, etc. The communicator 12 is able to transmit and receive various information (signal and data) to communicate with another apparatus (main terminal 2, supplementary display apparatus 3, etc.) connected through a communication network such as a local area network (LAN), wide area network (WAN), and the internet.

The storage 13 includes a nonvolatile semiconductor memory and a hard disk. The storage 13 stores various programs executed by the controller 11 and parameters necessary to perform the program.

The storage 13 stores data of the various display screens displayed on a main display 23 of the main terminal 2. Specifically, data used for screens such as an examination list screen S1 and an image confirmation screen S2 is stored.

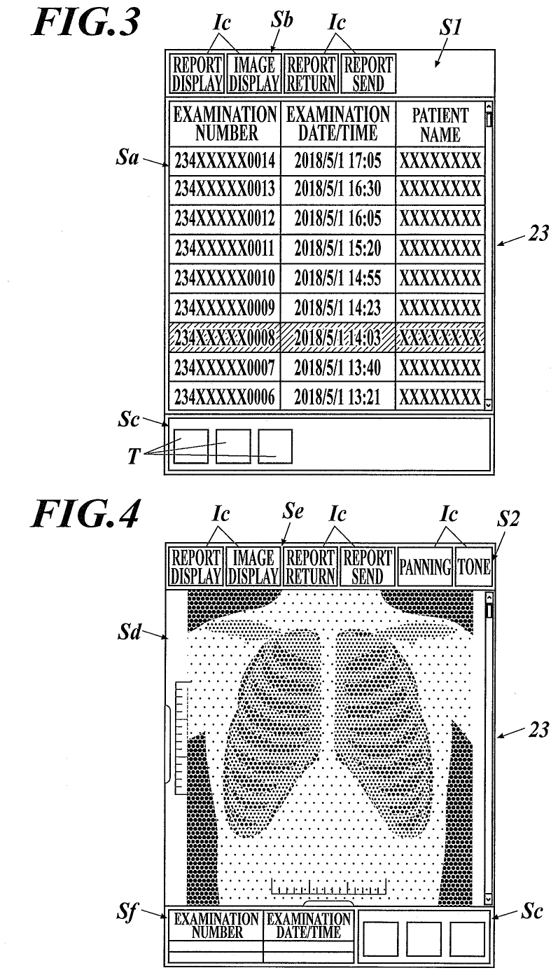

As shown in FIG. 3, the examination list screen S1 according to the present embodiment includes an examination list region Sa, an operation icon region Sb, and a thumbnail region Sc, etc.

The examination list region Sa is a region displaying as a list information such as examination number and examination date/time of a plurality of examinations performed in the past. In order to enhance the properties regarding searching by the user, only some digits of the examination number may be displayed.

The operation icon region Sb is a region displaying an icon Ic to perform various operations (for example, a report display icon to display a report of the examination selected from the examination list region Sa, an image display icon to switch to the image confirmation screen S2, a report return icon to display a report which is one report newer than the date/time that the report displayed on the supplementary apparatus is made, a report send icon which displays a report which is one report older than the date/time that the report displayed on the supplementary apparatus is made, and the like).

The thumbnail region Sc is a region displaying a thumbnail T corresponding to the examination selected from the examination list region Sa.

For example, as shown in FIG. 4, the image confirmation screen S2 according to the present embodiment includes an image display region Sd, an operation icon region Se, a related examination list region Sf, the thumbnail region Sc similar to the above-described examination list screen S1, and the like.

The image display region Sd is a region which displays the medical image corresponding to the examination selected in the examination list region Sa.

The operation icon region Se is a region which displays icon Ic (the following icons, operation icons similar to those which are displayed in the examination list screen S1, report display icon, report return icon, report send icon, and other icons such as examination list icon to switch to the examination list screen S1, panning icon to change the operation on the image displayed in the image display region to panning mode, a tone icon to change the operation on the image displayed in the image display region to tone changing mode, etc.) to perform various operations.

The related examination list region Sf is a region which displays a list of the other examinations that the subject of the displayed examination had.

The storage 13 includes a first medical information storage region and a second medical information storage region and the storage 13 stores first medical information in the first medical information storage region.

The first medical information according to the present embodiment includes the diagnostic medical image or the diagnostic date/time obtained by imaging the subject.

The storage 13 stores second medical information added to the first medical information stored in the second medical information storage region.

The second medical information according to the present embodiment includes at least any of the following, name of subject, ID number, birthdate, sex, opinion regarding corresponding first medical information, and diagnostic result based on corresponding first medical information.

The first medical information shows information with which it is difficult to specify the individual at one glance. The second medical information can be used to easily specify the individual compared to the first medical information. Therefore, the second medical information can be considered to be information which the patient does not want third parties to know.

The storage 13 according to the present embodiment stores display position information which corresponds the operation performed in the operating unit 24 of the main terminal 2 to the display position of the second medical information.

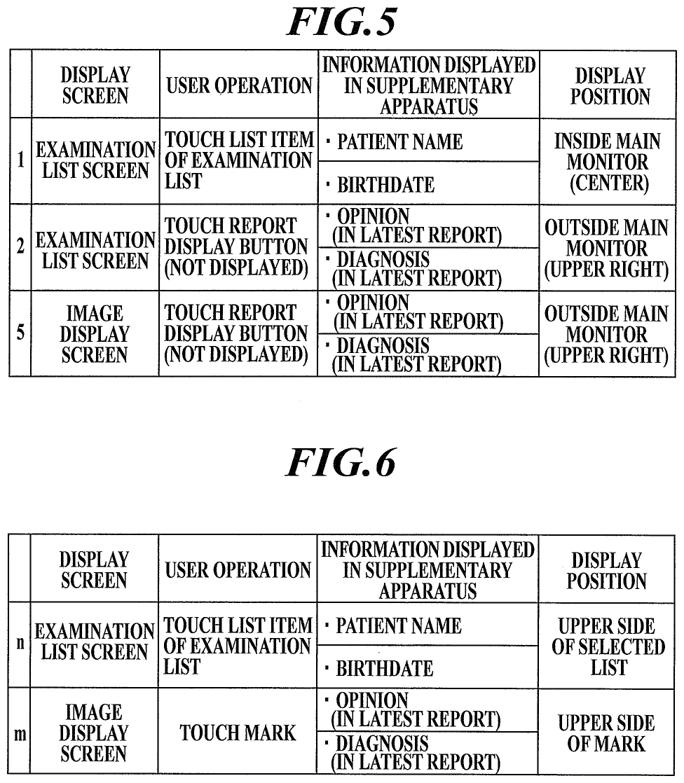

Specifically, for example, as shown in FIG. 5 and FIG. 6, the information is stored as a display position determination table which shows the relation among the display screen of the main display 23, the operation of the user, the displayed second medical information, and the display position of the second medical information.

Instead of providing the first medical information storage region and the second medical information storage region in one storage, the first medical information and the second medical information can be stored separately in two individual storages.

The controller 11 of the server 1 configured as described above includes the following functions which are performed by executing the programs stored in the storage 13.

For example, the controller 11 includes a function to select the first medical information displayed on the main display 23 described later from the plurality of first medical information stored in the storage 13 based on operation performed on the operating unit 24 of the main terminal 2, as described later.

Specifically, when the operation to call the examination list screen S1 is performed using the operating unit 24, the examination date/time which is the first medical information is selected.

When the operation to call the image confirmation screen S2 is performed using the operation unit 24, the medical image corresponding to the examination is selected.

The controller 11 includes the function to select the second medical information displayed on the supplementary display 33 from the plurality of second medical information stored in the storage 13 based on the first medical information displayed on the main display 23 and the operation performed on the operating unit 24 of the main terminal 2, described later.

Specifically, with reference to the display position determination table shown in FIG. 5 and FIG. 6, the first medical information which is displayed and the second medical information corresponding to the performed operation is selected.

The controller 11 according to the present embodiment includes a function to determine the display position of the second medical information in the supplementary display 33 of the supplementary display apparatus 3 based on the spatial information generated by the supplementary display apparatus 3 and the size of the main display 23 of the main terminal 2, as described later.

The method of determining the display position is described later.

[Main Terminal]

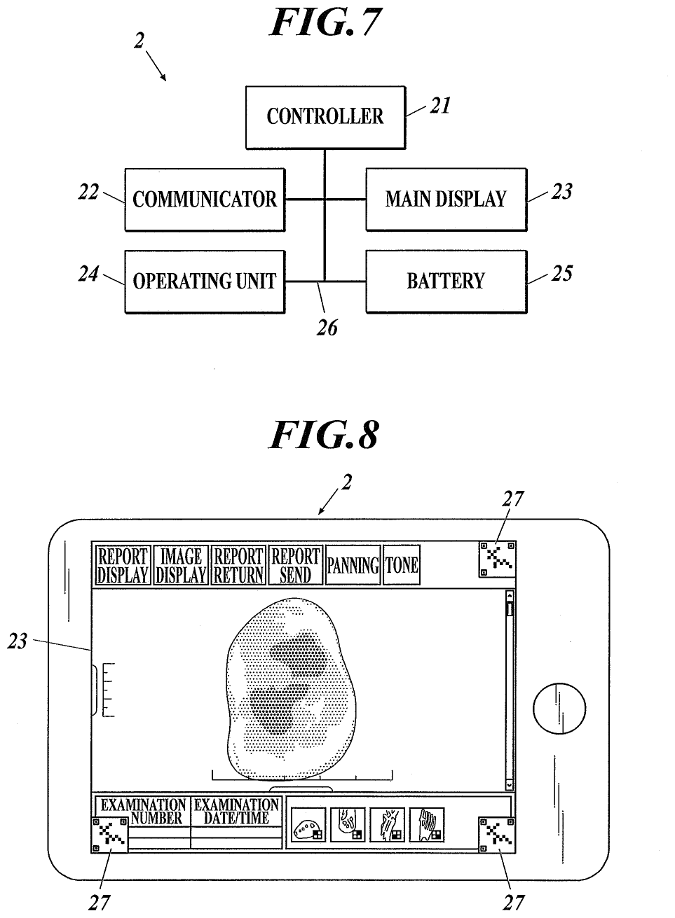

Next, the specific configuration of the main terminal 2 included in the display system 100 is described. FIG. 7 is a block diagram showing a specific configuration of the main terminal 2.

As shown in FIG. 7, the main terminal 2 includes a controller 21, a communicator 22, a main display 23, an operating unit 24, and an internal battery 25 which supplies power to the above units.

The above are connected by a bus 26.

The main terminal 2 according to the present embodiment includes a three-dimensional information transmitter 27.

The controller 21 includes a central processing unit (CPU), a random access memory (RAM), and the like. The CPU of the controller 21 obtains various programs stored in the storage 13 of the server 1 and deploys the program in the RAM. The various processes are executed according to the deployed program. The operation of each unit of the main terminal 2 is centrally controlled.

The communicator 22 includes a wireless module, etc. The communicator 22 is able to transmit and receive various information (signal and data) to communicate with another apparatus (server 1, supplementary display apparatus 3, etc.) connected through a communication network such as a local area network (LAN), wide area network (WAN), and the internet.

The main display 23 includes a monitor such as a liquid crystal display (LCD) and a cathode ray tube (CRT) and displays an image and various information according to the instruction of the display signal input from the controller 21.

The operating unit 24 can be operated by the user.

The operating unit 24 according to the present embodiment includes a touch panel layered on the main display 23 and operation corresponded with the coordinates of the display screen of the main display 23 is possible.

The operating unit 24 may include a plurality of buttons positioned two-dimensionally and a mouse.

The three-dimensional information transmitter 27 includes various sensors (position sensor and gyro sensor, for example) and a marker (for example, QR code (registered trademark)) provided on a surface of a housing of the main terminal 2.

Preferably, a plurality of markers are provided in some of the positions among the four corners of the main display 23. Alternatively, a plurality of markers are provided around the main display 23.

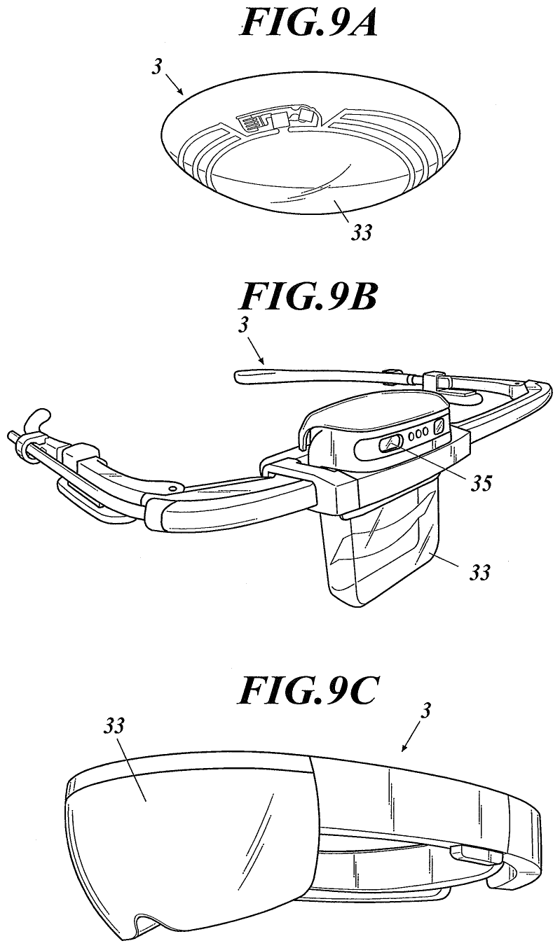

When the three-dimensional information transmitter 27 is the marker, for example, as shown in FIG. 8, the above is displayed on the main display 23.

The controller 21 of the main terminal 2 including the above structure performs the following functions by executing the program obtained from the server 1.

For example, the controller 21 obtains the data of the various display screens (examination list screen S1 and the image confirmation screen S2) stored in the server 1 and includes the function to display the display screens S1 and S2 based on the above data on the main display 23 as shown in FIG. 3 and FIG. 4.

The controller 21 includes the function to display the first medical information on the main display 23.

As described above, according to the present embodiment, the server 1 selects the first medical information based on the operation on the main terminal 2, and the controller 21 obtains and displays the first medical information selected by the server 1.

That is, when the examination list screen S1 is called, the examination date/time which is the first medical information is displayed in the examination list region Sa on the main display 23.

Alternatively, when the image confirmation screen S2 is called, the medical image which is the first medical information is displayed on the main display 23.

[Supplementary Display Apparatus]

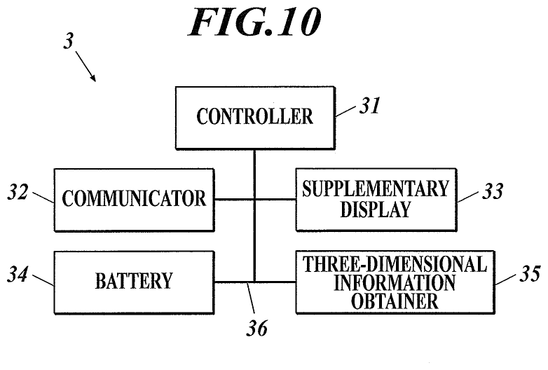

Next, the specific configuration of the supplementary display apparatus 3 included in the display system 100 is described. FIG. 9A, FIG. 9B, and FIG. 9C are diagrams showing an example of the supplementary display apparatus 3 and FIG. 10 is a block diagram showing the specific configuration of the supplementary display apparatus 3.

The supplementary display apparatus 3 according to the present embodiment includes a supplementary display 33 including a transparent display.

Specifically, the display may be a contact lens type display attached to the eye as shown in FIG. 9A, a head mount display attached to the head as shown in FIG. 9B and FIG. 9C or any other wearable display attached to other portions.

The following devices which do not include the transparent display may be used, for example, a head mount display including a camera which can image surrounding images including the main terminal 2 by attaching to the head and a nontransparent display which displays the imaged surrounding images, or a watch type display which is basically difficult for those other than the user wearing the device to see.

Therefore, when the user wears the supplementary display apparatus 3, it is difficult for those other than the user wearing the supplementary display apparatus 3 to see the supplementary display 33.

Other than devices directly worn by the user as shown in FIG. 9A, FIG. 9B, and FIG. 9C, the supplementary display apparatus 3 may be a device used by attaching to an accessory (for example, glasses G) worn by the user, as shown in FIG. 1.

For example, other than the above-described supplementary display 33, as shown in FIG. 10, the supplementary display apparatus 3 includes a controller 31, a communicator 32, and an internal battery 34 which supplies power to the above units.

The supplementary display apparatus 3 according to the present embodiment includes a three-dimensional information obtainer 35.

The above units are connected to each other by a bus 36.

The controller 31 includes a central processing unit (CPU), a random access memory (RAM), and the like. The CPU of the controller 31 obtains various programs stored in the storage 13 of the server 1 and deploys the program in the RAM. The various processes are executed according to the deployed program. The operation of each unit of the supplementary display apparatus 3 is centrally controlled.

The communicator 32 includes a wireless module, etc. The communicator 32 is able to transmit and receive various information (signal and data) to communicate with another apparatus (server 1, main terminal 2, etc.) connected through a communication network such as a local area network (LAN), wide area network (WAN), and the internet. That is, the communicator is able to receive various data directly from the server 1. Alternatively, the communicator is able to receive various data through the main terminal 2.

When various data is received through the main terminal 2, it is possible to set on the server 1, the main terminal 2 or the supplementary display apparatus 3 not to display on the main terminal 2 the information displayed on the supplementary display apparatus 3.

The three-dimensional information obtainer 35 includes various sensors (position sensor, gyro sensor, etc.) and a camera with which the user wearing the supplementary display apparatus 3 can image an object (main terminal 2) in the direction that the user is viewing.

The controller 31 of the supplementary display apparatus 3 including the above structure performs the following functions by executing the programs obtained from the server 1.

For example, the controller 31 includes the function to display on the supplementary display 33 the second medical information related to the first medical information displayed on the main display 23.

As described above, according to the present embodiment, the server 1 selects the second medical information based on the first medical information displayed on the main terminal 2 and the operation performed on the main terminal 2, and the controller 31 obtains and displays the second medical information selected by the server 1.

According to the present embodiment, the second medical information is displayed on the supplementary display 33 in the display position determined by the server 1.

The “display position” is described later.

The controller 31 includes a function to generate at least one type of spatial information among relative position, direction, and distance of the main display 23 in the main terminal 2 with relation to the supplementary display apparatus 3 using the three-dimensional information transmitter 27 of the main terminal and the three-dimensional information obtainer 35.

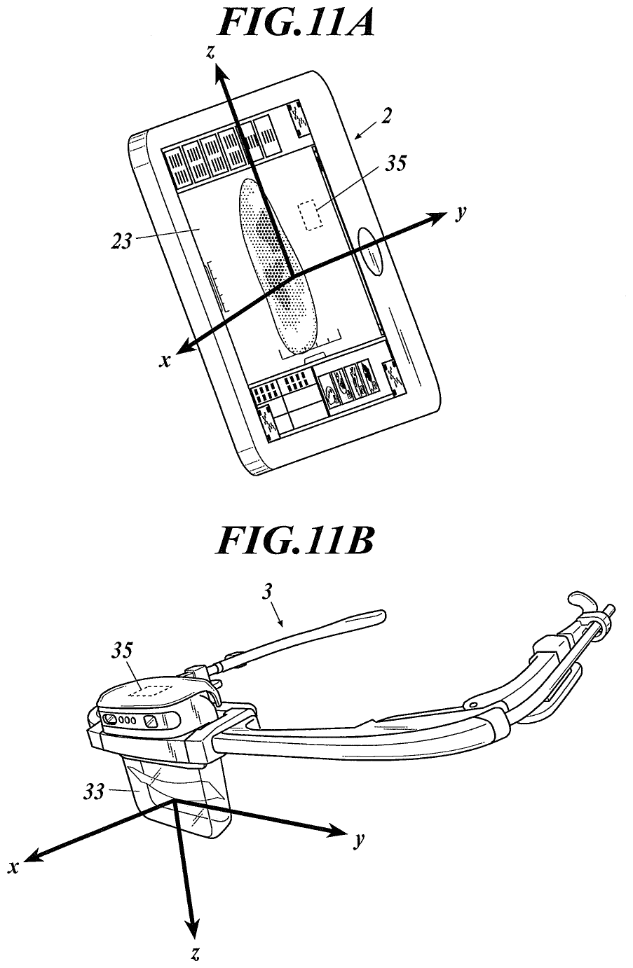

When the three-dimensional information obtainer 35 includes a sensor, as shown in FIGS. 11A and 11B, the three-dimensional information generated by the three-dimensional information transmitter 27 (sensor on the main terminal side) and the three-dimensional information generated by the three-dimensional information obtainer 35 (sensor) are obtained and the above are analyzed. With this, the relative position and the direction that the screen faces are calculated for the main terminal 2 with relation to the supplementary display apparatus 3. The calculated relation of the positions is to be the spatial information.

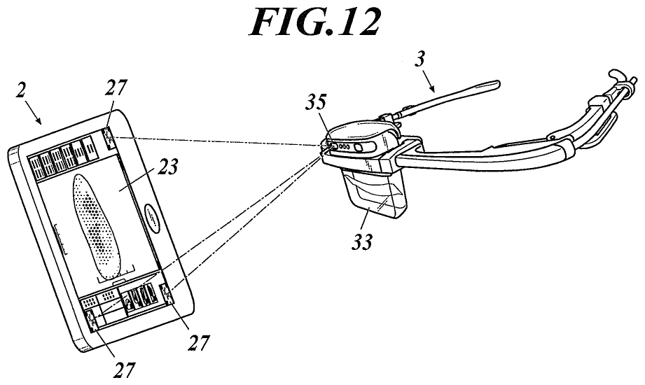

When the three-dimensional information obtainer 35 includes a camera, for example, as shown in FIG. 12, the main terminal 2 including the three-dimensional information transmitter 27 (marker) is imaged. Based on the position and the size of the three-dimensional information obtainer 27 in the obtained image, the relative position and the direction that the screen faces are calculated for the main terminal 2 with relation to the supplementary display apparatus 3. The calculated relation of the positions is to be the spatial information.

The supplementary display apparatus 3 is attached to the user, and therefore, the relative position, direction, and distance of the main display 23 in the main terminal 2 with relation to the supplementary display apparatus 3 keep changing according to the movement of the user. Therefore, the controller 31 repeatedly generates the above-described spatial information while the second medical information is displayed on the supplementary display 33.

[Display Position]

Next, the function which determines the display position of the above-described server 1 is described in detail.

The controller 11 of the server 1 according to the present embodiment includes a function to determine the display position of the second medical information on the supplementary display 33 of the supplementary display apparatus 3 based on the spatial information generated by the supplementary display apparatus 3 and the size of the main display 23 of the main terminal 2.

Specifically, the display position is determined so that the second medical information can always be seen in a position determined with relation to the first medical information. That is, the display position is determined so that the second medical information overlaps on the portion surrounding the main display 23 in the casing of the main terminal 2, the surrounding edge portion of the main display 23, or the inner side of the main display 23.

In addition to using the spatial information and the size of the main display 23, the controller 11 includes the function to determine the display position of the second medical information on the supplementary display 33 of the supplementary display apparatus 3, based on the display position information corresponding to the operation on the operating unit 24 of the main terminal 2.

Specifically, the display position determining table is referred each time an operation on the operating unit 24 of the main terminal 2 by the user is detected, and the display position is determined based on the display position information corresponding to the performed operation.

As described above, according to the present embodiment, the display position information is stored in the form of a display position determination table as shown in FIG. 5 and FIG. 6. The display position information includes the relative display position with relation to the main display 23 of the main terminal 2, and the relative display position with relation to the coordinates corresponding to the operation on the operating unit 24.

The “relative display position with relation to the main display 23” is a position such as inner side (for example, center, etc.) or outer side (for example, upper right, etc.) of the main display 23 as shown in FIG. 5.

The “relative display position with relation to the coordinates” is a position such as upper side of the coordinates where the selected examination item It exists or upper side of the coordinates where the mark M exists as shown in FIG. 6.

Instead of specifying the display position with the direction, the display position may be specified with a pixel number or distance.

Then, when the display position information corresponding to the performed operation is a relative display position with relation to the main display 23, the controller 11 determines the inner side or the outer side of the main display 23 as the display position of the second medical information for the selected subject based on the generated spatial information.

When the display position information corresponding to the operation is the relative display position with relation to the coordinates, the surrounding portion (upper side) of the examination item It or the mark M positioned on the coordinates is determined as the display position of the second medical information of the selected subject based on the coordinates corresponding to the operation of the user on the operating unit 24 and the spatial information.

As described above, the relative position, direction, and distance of the main display 23 in the main terminal 2 with relation to the supplementary display apparatus 3 keeps changing according to the movement of the user, and therefore, the determination of the display position is performed repeatedly using the spatial information generated repeatedly.

[Operation of Medical Information Display System]

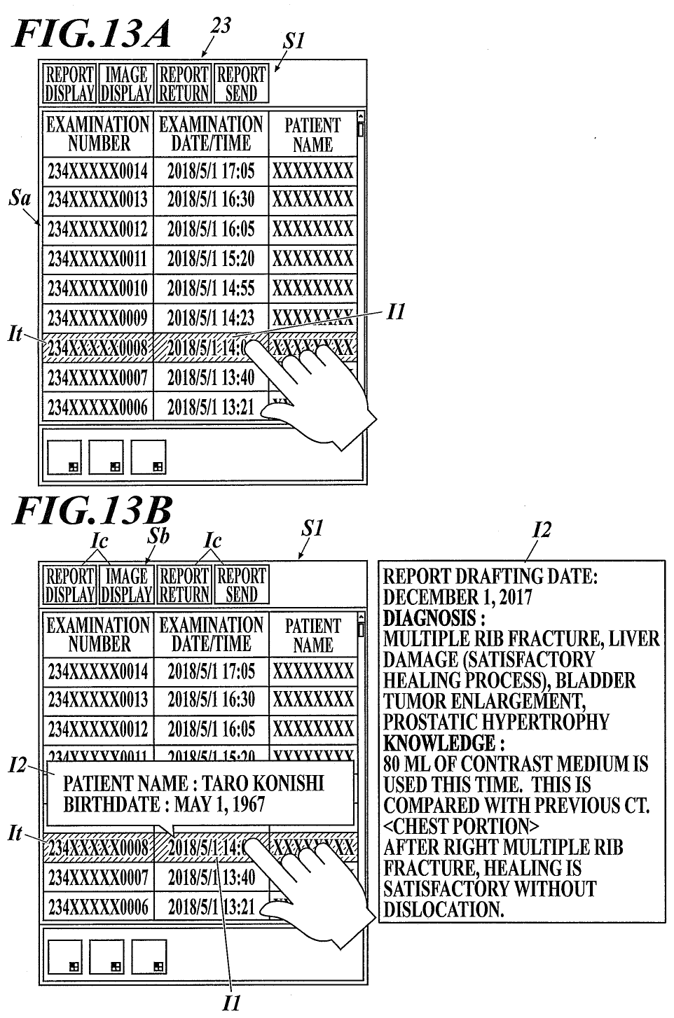

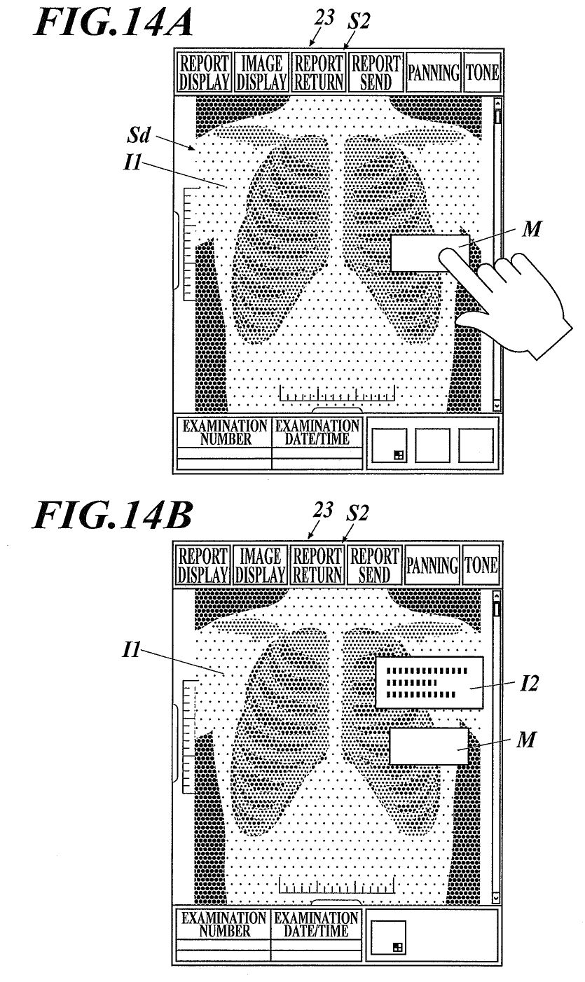

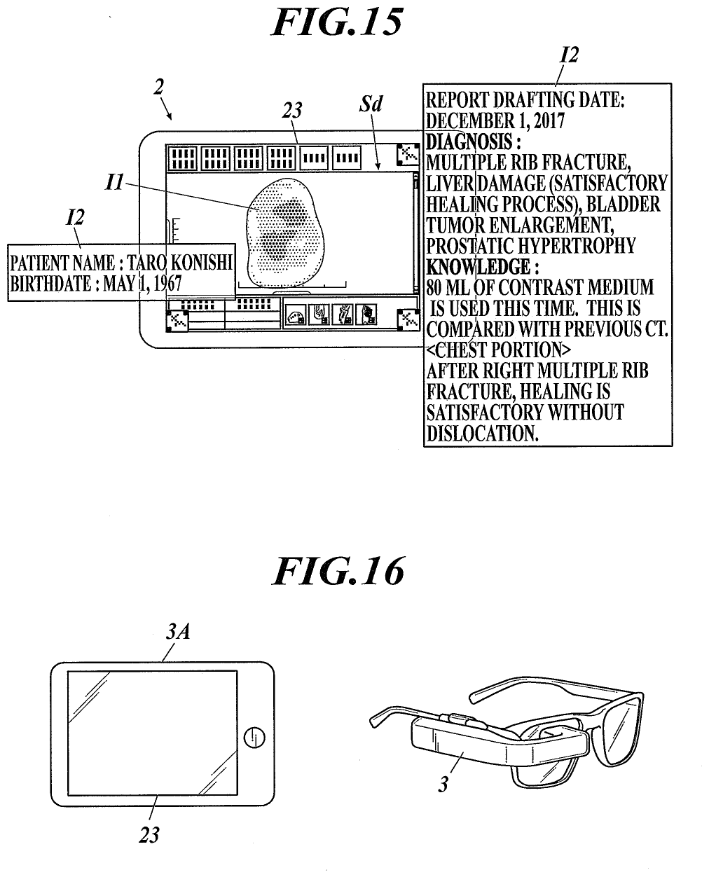

Next, the operation of the display system 100 is described. FIG. 13A, FIG. 13B, FIG. 14A, FIG. 14B, and FIG. 15 are diagrams showing how the main terminal 2 can be viewed from the user wearing the supplementary display apparatus 3.

When the user wearing the supplementary display apparatus 3 touches (operates the operating unit 24) the examination item It which the user desires to view from the examination list region Sa while looking at the main display 23 of the main terminal 2 on which the examination list screen S1 (plurality of examination date/time) is displayed as shown in FIG. 13A, personal information (second medical information I2) such as the name of the corresponding subject and the birthdate is displayed as shown in FIG. 13B.

When one examination is selected and the user touches the report display icon IC of the operation icon region Sb, the opinion (second medical information I2) regarding the corresponding medical image (first medical information I1) and the diagnostic result (second medical information I2) regarding the corresponding medical image I1 are displayed on the side of the main display 23 of the main terminal 2.

When the user touches the image display icon IC when one examination is selected on the examination list screen S1, as shown in FIG. 14A and FIG. 15 for example, the display screen of the main display 23 switches to the image confirmation screen S2, and the medical image I1 of the corresponding subject is displayed on the image display region Sd (center of main display 23).

Here, when the mark M in the medical image I1 is touched, the corresponding second medical information I2 is displayed on the upper side of the mark M as shown in FIG. 14B or is displayed on the side of the medical image I1 as shown in FIG. 15.

When the user touches the examination list icon IC when the image confirmation screen S2 is displayed, the display screen of the main display 23 switches to the examination list screen S1 as shown in FIG. 13A, and the examination can be selected again.

According to the present embodiment, the supplementary display 33 of the supplementary display apparatus 3 includes a transparent display. Therefore, even if the second medical information is displayed on the supplementary display 33, the user is able to confirm by sight the main terminal 2 through the supplementary display 33.

According to the present embodiment, the display position of the second medical information in the supplementary display 33 changes according to the spatial information.

Therefore, as shown in FIG. 13B, FIG. 14B, and FIG. 15, the user wearing the supplementary display 33 is able to confirm by sight the second medical information I2 in the state always positioned in the surroundings of the first medical information I1 (surroundings of the main display 23 in the main terminal 2, position overlapping with a portion of the main display 23 or inner side of the main display 23 (surroundings of examination item It or mark M)).

That is, it is possible to view the medical information in a form of augmented reality (AR) virtually displaying the second medical information around the first medical information.

When the communication between the main terminal 2 and the supplementary display apparatus 3 is synchronized for information such as the mark M and the second medical information I2 which are displayed on the supplementary display apparatus 3 but which are not actually displayed on the main terminal 2, the display position of the mark M and the second medical information I2 can be changed as if displayed on the main terminal 2 according to operation by the user on the main terminal 2.

According to such configuration, when the position of the mark M and the second medical information I2 move outside the main terminal 2, the operation cannot be performed on the main terminal 2. Then, it is possible to perform operation to place the above back on the main terminal 2 using a button on the main terminal 2 (not shown) or the operating unit (not shown) on the supplementary display apparatus 3.

As described above, the display system 100 according to the present embodiment includes a server 1 including a storage 13 which stores first medical information and second medical information which is added to the first medical information, a main terminal 2 including a main display 23 and a controller 21 which obtains the first medical information from the server 1 to be displayed on the main display 23, and a supplementary display apparatus 3 including a supplementary display unit 33 which the user is able to wear and which can be confirmed by sight by the user wearing the unit, and a controller 31 which obtains the second medical information I2 corresponding to the first medical information I1 displayed on the main display 23 from the server 1 and which displays the above on the supplementary display 33.

Therefore, by setting information which the subject does not want third parties to know, for example, personal information of the subject as second medical information, the user can perform diagnosis in facilities outside medical facilities where a large number of unspecified people exist without leaking to third parties medical information which the subject does not want third parties to know.

When all medical information is displayed on the head mount display, the instruction to display the medical information needs to be performed by gestures or remote control. With such operation, there is the problem that such operation cannot be performed in small spaces. Further, the user operation may be unpleasant to third parties. Therefore, such operation is not suitable for public places where many people gather.

However, according to the present embodiment, the instruction for display on the main terminal 2 and display on the supplementary display apparatus 3 can be performed only by operation (screen touch) on the main terminal 2. Therefore, it is possible to prevent problems as described above.

### Second Embodiment

Next, the medical information display system (hereinafter referred to as display system 100A) according to the second embodiment of the present invention is described with reference to FIG. 16.

Here, the same reference numerals are applied to the structures which are the same as the first embodiment, and the description is omitted.

The display system 100 according to the first embodiment includes a server 1. However, as shown in FIG. 16, the medical information display system 100A according to the present embodiment does not include a structure which corresponds to the server 1 according to the first embodiment.

The main terminal 2A includes a controller with a higher processing ability than the main terminal 2 according to the first embodiment. The main terminal 2A is provided with a storage which stores programs to realize various functions included in the server 1 according to the first embodiment.

The supplementary display apparatus 3 is the same as the first embodiment.

Alternatively, the main terminal according to the present embodiment can be the same as the main terminal 2 according to the first embodiment, and the various functions included in the server 1 of the first embodiment can be included in the supplementary display apparatus or the various functions included in the server 1 can be divided between the main terminal and the supplementary display apparatus.

According to the configuration of the medical information display system 100A of the present embodiment, similar to the first embodiment, the user can perform diagnosis without leaking to third parties the medical information which the subject does not want third parties to know even outside medical facilities where a large number of unspecified people exist.

The present invention is described in the embodiments above, but the present invention is not limited to the embodiments above, and suitable changes can be made without departing from the scope of the invention.

For example, according to the above-described embodiments, the transparent display is used as the supplementary display 33 of the supplementary display apparatus 3. However, a transparent display as shown in FIG. 1 does not have to be used as the supplementary display 33. In this case, the necessity to change the position of displaying the second medical information depending on the relation of the positions between the main terminal 2 and the supplementary display apparatus 3 is small. Therefore, functions such as generating spatial information or determining the display position can be omitted.

According to the above-described embodiments, name of subject, ID number, birthdate, sex, opinion regarding corresponding first medical information, and diagnostic result based on corresponding first medical information are included in the second medical information. However, some of the above information can be displayed on the main terminal 2 as the first medical information.

Based on the operation on the operating unit 24 of the main terminal 2, it is possible to select whether to display the second medical information on the main terminal 2 or the supplementary display apparatus 3.

Among the medical information displayed on the main terminal 2 and the supplementary display apparatus 3, the user can operate the operating unit 24 so that the medical information which is no longer necessary is not displayed.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-112394 filed on Jun. 13, 2018 is incorporated herein by reference in its entirety.

What is claimed is:

1. A medical information display system comprising:
a server which includes a storage which stores first medical information and second medical information added to the first medical information;
a main terminal which includes a main display and a first hardware processor which obtains the first medical information from the server to display the first medical information on the main display; and
a supplementary display apparatus which includes a supplementary display which can be worn by the user and which can be confirmed by sight by the user wearing the supplementary display apparatus and a second hardware processor which obtains the second medical information related to the first medical information displayed on the main display from the server to display the second medical information on the supplementary display.

2. The medical information display system according to claim 1, wherein,
the main terminal includes an operating unit which can be operated by the user,
the server includes a third hardware processor in which the first medical information displayed on the main display is selected from a plurality of sets of first medical information stored in the storage based on an operation on the operating unit and in which the second medical information displayed on the supplementary display is selected from a plurality of sets of second medical information stored in the storage based on the first medical information displayed on the main display and the operation on the operating unit,
the first hardware processor obtains the first medical information selected by the third hardware processor to display the first medical information on the main display, and
the second hardware processor obtains the second medical information selected on the third hardware processor to display the second medical information on the supplementary display.

3. The medical information display system according to claim 1, wherein,
the supplementary display apparatus includes a transparent display as the supplementary display,
the second hardware processor generates spatial information among at least any of a relative position, direction or display of the main display with relation to the supplementary display apparatus,
the second hardware processor determines a display position of the second medical information on the supplementary display based on the generated spatial information and a size of the main display, and
the second hardware processor displays the second medical information on the determined display position.

4. The medical information display system according to claim 3, wherein,
the storage stores display position information in which an operation on the operating unit is corresponded to the display position of the second medical information, and
based on the display position information corresponding to the operation on the operating unit, the second hardware processor determines a display position of the second medical information on the supplementary display.

5. The medical information display system according to claim 4, wherein the display position information includes a relative display position with relation to the main display.

6. The medical information display system according to claim 4, wherein,
an operation corresponded to coordinates on a display screen of the main display is possible on the operating unit, and
the display position information includes the relative display position with relation to the coordinates corresponding to the operation on the operating unit.

7. The medical information display system according to claim 1, wherein the first medical information includes a medical image obtained by imaging a subject or a date and time of examination.

8. The medical information display system according to claim 1, wherein the second medical information includes at least any of the following, name of subject, ID number, birthdate, sex, opinion regarding corresponding first medical information, and diagnostic result based on corresponding first medical information.
